(19) United States
(12) Patent Application Publication
(10) Pub. No.: US 2006/0136047 A1
(43) Pub. Date: Jun. 22, 2006

(54) Tissue graft prosthesis devices containing juvenile or small diameter submucosa

(76) Inventors: F. Joseph Obermiller, West Lafayette, IN (US); Michael C. Hiles, Lafayette, IN (US); Jason P. Hodde, West Lafayette, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 10/523,892

(22) PCT Filed: Sep. 4, 2003
(86) PCT No.: PCT/US03/27695

§ 371 Date: Sep. 15, 2005

Related U.S. Application Data
(60) Provisional application No. 60/408,914, filed on Sep. 6, 2002.

(51) Int. Cl. A61F 2/06

(52) U.S. Cl. 623/1.41; 623/1.44

(57) ABSTRACT

Described are preferred tissue graft materials that incorporate juvenile submucosa tissue from a warm-blooded vertebrate. Preferred materials incorporate juvenile small intestinal submucosa tissue from a mammal such as a porcine mammal, and the constructs are preferably in tubular form and utilized the isolated submucosa tissue in its native, intact tubular form. More preferred devices are multi-laminate and include the juvenile submucosa tissue in addition to at least one, and preferably several other layers providing increased strength or other advantageous properties to the construct.

### SUMMARY OF THE INVENTION

Accordingly, one aspect of the present invention provides a tubular graft prosthesis that includes a tubular element having walls and a lumen. The walls of the prosthesis include juvenile submucosa tissue from a warm-blooded vertebrate. In preferred forms, the juvenile submucosa tissue retains a natural, intact tubular form, especially having a diameter not exceeding 12 mm. The juvenile submucosa can be positioned in the prosthesis so as to provide the innermost layer, and thereby define the surface of the lumen. More preferred prosthesis devices of the invention will include at least one additional wall layer which can, for example, be another layer of collagenous tissue such as juvenile or adult submucosa tissue. One or more layers of a synthetic material may also be provided.

In another embodiment, the invention provides a tissue graft composition that in includes juvenile submucosa tissue of a warm-blooded vertebrate.

In another embodiment, the invention provides a small-diameter tubular graft construct that includes a tubular element having walls defining a lumen. The walls include at least a first layer formed with an intact tubular submucosa segment having a native internal diameter no greater than about 12 mm. The walls in such constructs may also include one or more additional layers formed with tissue materials and/or synthetic polymer materials as described further hereinbelow.

In still another aspect, the present invention provides a method for tissue grafting in an animal that comprises grafting the animal with a tissue graft material including juvenile submucosa tissue from a warm-blooded vertebrate.

The present invention provides improved and alternative tissue graft prosthesis devices including tubular graft constructs, and manufacturing and grafting methods involving the same. Additional embodiments and features and advantages of the invention will be apparent from the descriptions herein.

### DESCRIPTION OF THE DRAWINGS

FIG. 1 provides a perspective view of a tubular graft prosthesis device of the present invention.

FIG. 2 provides a cross-sectional view of the tubular graft prosthesis device depicted in FIG. 1 taken along line 2-2 and viewed in the direction of the arrows.

FIGS. 3A-3G depict steps used in the manufacture of a 5-layer tubular prosthesis device of the invention.

FIG. 4 depicts a tubular submucosa covered stent device in accordance with the invention.

### DETAILED DESCRIPTION

For the purpose of promoting an understanding of the principles of the invention, reference will now be made to certain embodiments thereof and specific language will be used to describe the same. It will nevertheless be understood that no limitation of the scope of the invention is thereby intended, such alterations, further modifications and applications of the principles of the invention as described herein being contemplated as would normally occur to one skilled in the art to which the invention relates.

As disclosed above, one aspect of the present invention provides tissue graft prosthesis devices that incorporate juvenile submucosa tissue from a warm-blooded vertebrate. Preferred graft devices include a tubular element having walls and lumen, wherein the walls include juvenile submucosa tissue or small-diameter tubular submucosa tissue from a warm-blooded vertebrate. In this regard, as used herein, the term “juvenile” refers to a warm-blooded vertebrate having an age of not greater than about 30 days. This includes both fetal (prenatal) submucosa tissues and those taken from postnatal animals. The term “small diameter” as used herein refers to tubular materials having an internal diameter no greater than about 12 mm. For example, small diameter intestinal submucosa may be obtained from juvenile animals, and/or from older (including adult) animals of dwarf, pigmy, or other unusually small breeds. As well, the animal from which the submucosa tissue is taken may be male or female.

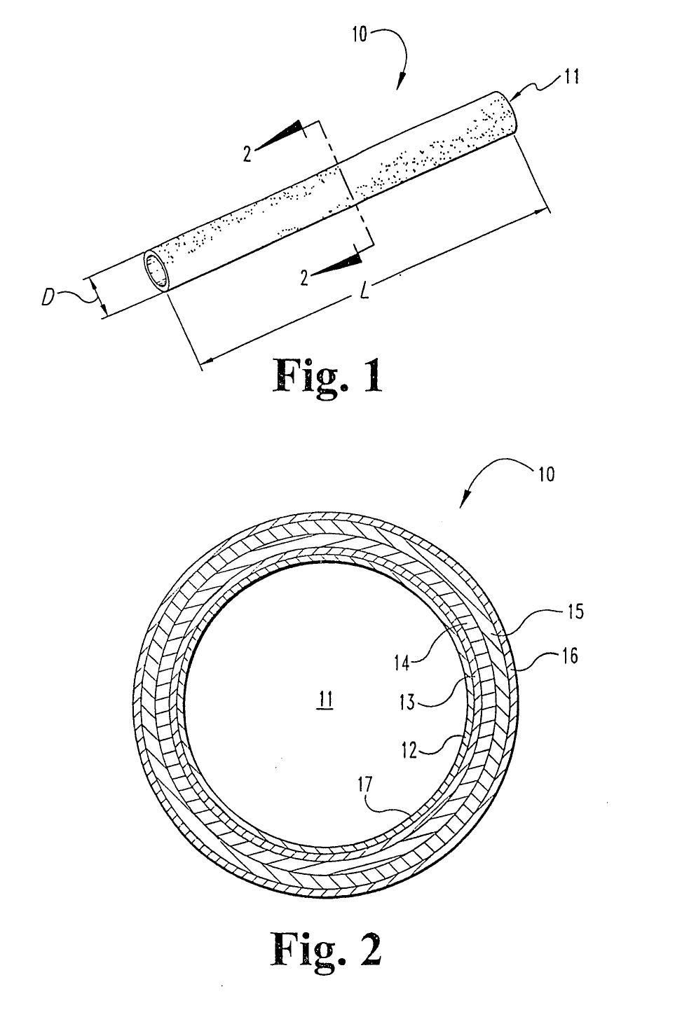

With reference now to FIG. 1, shown is a perspective view of a tubular graft prosthesis 10 in accordance with the present invention. Tubular graft prosthesis 10 defines an inner lumen 11 and has a length L and diameter D rendering the construct suitable for the intended use, for example a vascular use.

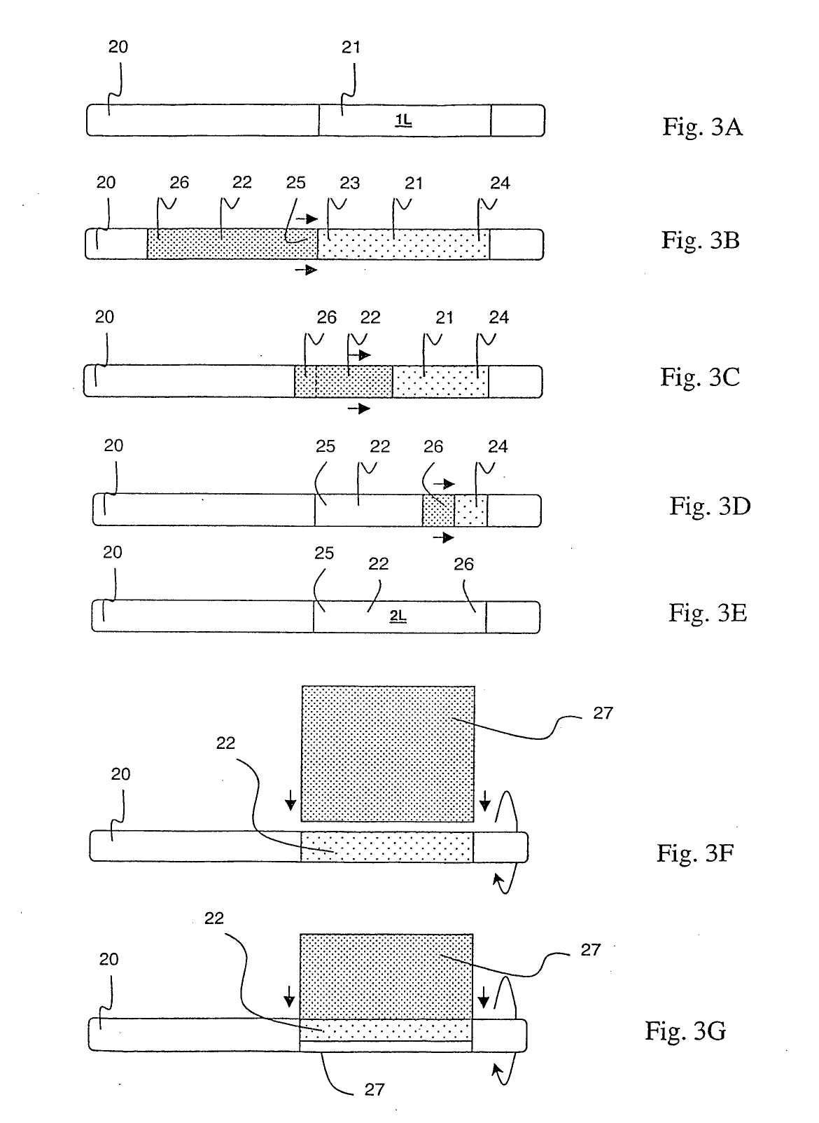

With reference now to FIGS. 1 and 2 together, shown in FIG. 2 is a cross-sectional view of the prosthesis 10 of FIG. 1 taken along line 2-2 and viewed in the direction of the arrows. Prosthesis 10 has walls defining inner lumen 11, preferably including several layers of material as illustrated. In particular, shown in prosthesis 10 is a first tubular layer 12, a second layer tubular layer 13, a third tubular layer 14, a fourth tubular layer 15, and a fifth tubular layer 16. In accordance with certain aspects of the invention, at least one of these layers includes juvenile submucosa from a warm-blooded vertebrate animal, or otherwise includes an intact tubular submucosa segment having a small native internal diameter (12 mm or less). The animal is preferably a mammal, such as a porcine, ovine, bovine, or other mammalian animal. Human donor tissues may also be used in the present invention. In the case of juvenile porcine submucosa, the animal at harvest will typically not exceed about 10 kilograms (kg).

In preferred aspects of the invention, the juvenile or other small diameter submucosa tissue will retain its intact, tubular form as harvested from the animal. More preferably, at least the innermost layer 12 will be formed from intact, tubular juvenile submucosa tissue. In this fashion, the surface 17 of the lumen 11 will be defined by the intact juvenile submucosa tissue, and will be free of any seams that would otherwise be created when configuring sheet-form tissue into a tube. Preferred devices will include at least one additional layer, for example, layers 13, 14, 15 and 16 as illustrated in FIG. 2. These additional layers can be made of any suitable material and desirably provide reinforcement and strength to the device supplemental to that provided by innermost layer 12. When innermost layer 12 is comprised of juvenile submucosa tissue, one or more of layers 13, 14, and 15 may, for example, be formed of synthetic materials such as synthetic polymer materials. Suitable synthetic materials may be biodegradable or non-biodegradable materials. These include, for example, synthetic biocompatible polymers such as cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene, polytetrafluoroethylene, or mixtures or copolymers thereof; polylactic acid, polyglycolic acid or copolymers thereof, a polyanhydride, polycaprolactone, polyhydroxy-butyrate valerate, polyhydroxyalkanoate, or another biodegradable polymer.

In certain embodiments of the invention where layer 12 is comprised of juvenile submucosa, one or more of, and potentially all of layers 13, 14, 15 and/or 16 are formed from additional collagenous materials. For example, suitable collagenous materials include extracellular matrix layers including, for instance, submucosa, renal capsule membrane, dura mater, pericardium, serosa, peritoneum or basement membrane layers, including liver basement membrane. These layers may be isolated and used as intact membranes, or reconstituted collagen layers including collagen derived from these materials or other collagenous materials may be used.

Desirably, layers 13, 14, 15 and 16 are made from additional submucosa tissue layers. Suitable submucosa tissues for these purposes include, for instance, intestinal submucosa including small intestinal submucosa, stomach submucosa, urinary bladder submucosa, and uterine submucosa. Small intestinal submucosa, when employed, can be used in an intact, native tubular form or can be a tubular form shaped from flat sheets including one or more seams along all or a portion of its length. Desirably, at least one of layers 13, 14, 15 and 16 will include adult submucosa tissue, as such tissue in its native condition is generally superior in mechanical properties to juvenile submucosa tissue. In this fashion, adult submucosa tissue can be used to provide strength to the overall graft construct 10. Porcine small intestinal submucosa is particularly preferred for these purposes.

In one form, intermediate layers 14 and 15 can be made from adult small intestinal submucosa, and intermediate layer 12 and outermost layer 16 can be made from juvenile small intestinal submucosa, preferably again in its native, intact tubular form. In this fashion, seamless inner layer 12 and seamless outer layer 16 can be provided.

Layers 12, 13, 14, 15 and 16 can be adhered to one another so as to generally form a unitary construct. This adherence may be achieved, for example, by crosslinking, including for example dehydrothermal crosslinking or chemical crosslinking, and/or by the use of a bonding agent. As bonding agents for these purposes, one may use fibron glue, or gelatin or collagenous pastes in sufficient amount to bond adjacent layers to one another.

Tubular devices of the invention may be prepared, for example, by positioning the appropriate tissue layers over a mandrel, and subsequently bonding or adhering the tissue layers together to form a generally unitary tubular construct. This may be accomplished, for instance, using intact tubes, and/or by wrapping or winding sheet- or strip-form adult submucosa tissue around the mandrel to form overlapped sections which are subsequently bonded or adhered. In some embodiments, an outermost covering layer may be provided by an intact juvenile submucosa segment positioned over the underlying tissue layers. If a bonding agent is to be used in forming the construct, the agent or its components can be applied at appropriate points intermediate the application of layers to the mandrel. The entire construct can then be dried, e.g., lyophilized and/or dried under vacuum, to form the overall tubular graft construct.

In some embodiments of the invention, tubular prosthesis devices are prepared using a two component bonding agent such as fibrin glue (e.g., having thrombin and fibrinogen as separate components). To prepare such devices, subsequent layers are added after coating the previously-applied layer with a first component of the bonding agent (e.g., thrombin) and coating a layer to be applied with a second component of the bonding agent (e.g., fibrinogen). Thereafter, the layer to be applied is positioned over the previously-applied layer so as to bring the two bonding components into contact, thus causing the curing process to begin. This process can be repeated for any and all additional layers to be applied to the tubular construct.

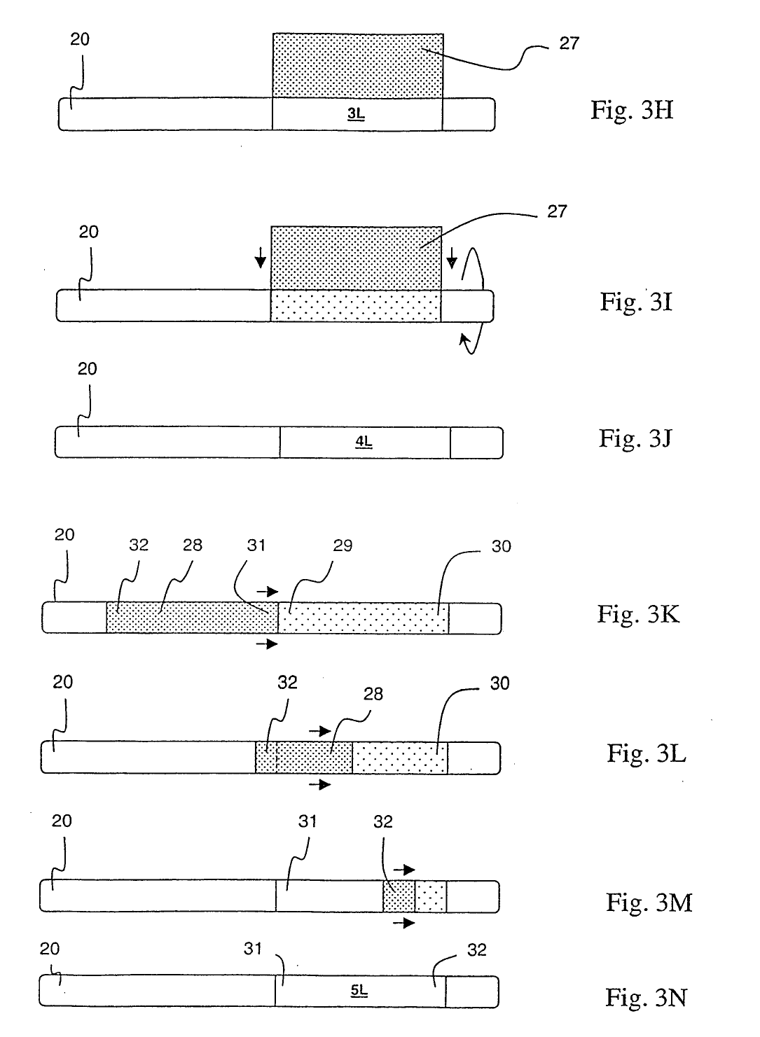

With reference now to FIGS. 3A through 3G, an illustrative manufacture of a 5-layer (5L) tubular device of the invention will now be described. An intact tubular submucosa segment 21 from a juvenile animal may first be positioned over a mandrel 20 as depicted in FIG. 3A, to provide a one-layer (1L) construct. Thrombin (light shading, FIG. 3B) is then applied to the intact segment 21. A second intact tube of juvenile submucosa 22 is provided either on an extension of the same mandrel as illustrated, or on a second mandrel connectable to the first mandrel. The second intact segment 22 is coated with fibrinogen (dark shading), and the segment 22 is positioned immediately adjacent the first segment 21. Segment 22 is then and pulled over the first submucosa tube in a fashion causing inversion of the tube 22 (FIGS. 3C-3E). Thus, leading end 25 of segment 22 remains substantially in place, but inverted, in contact with the trailing end 23 of segment 21. Trailing end 26 of segment 22 finally inverts and comes into contact with leading end 24 of the first segment 21. In this fashion, portions of the two submucosa tubes coming together will remain substantially together, i.e., one, submucosa layer will not pulled along another submucosa layer. This is beneficial in that as the bonding agent begins to cure, movement of submucosa layers relative to one another becomes difficult. The inversion of the second tube is continued until it is completely inverted and lying atop the first submucosa tube, creating a two-layer (2L) construct as illustrated in FIG. 3E. The fibrin glue of the 2L construct is then allowed to cure (typically 1 to 5 minutes). The outer surface of the 2L construct is coated with thrombin (light shading, FIG. 3F). Fibrinogen (dark shading, FIG. 3F) is then applied to one surface of a sheet 27 of adult submucosa of a dimension sufficient to encircle the prior-applied layers two times. As illustrated in FIGS. 3F-3H, the adult submucosa is then wrapped around the 2L construct for a single turn (clockwise rotation in FIGS. 3F-3G), resulting in a completed three-layer (3L) construct on the mandrel 20. During or after curing of the applied fibrin glue components, thrombin is applied to the outer surface of the 3L construct (light shading, FIG. 3I). A second turn of the adult submucosa sheet 27 is then completed (FIG. 3I), bringing the applied thrombin and fibrinogen components into contact with one another, and forming the completed four-layer device (4L, FIG. 3J). During or after cure of the newly-contacted fibrin glue components, a third intact tubular juvenile submucosa segment 28 (FIG. 3K) is positioned adjacent to the applied layers, thrombin (light shading) is coated onto the 4L construct and fibrinogen (dark shading) is coated onto the third tubular segment 28. The third tubular segment 28 is then pulled and inverted over onto the applied layers (FIGS. 3K-3M) as before to complete the 5L construct (FIG. 3N), with the leading end 31 of the third segment 28 lying atop the trailing end 29 of segment the 4L construct, and the trailing end 32 of segment 28 lying atop the leading end 30 of the 4L construct. The device is then allowed to cure and is trimmed as necessary. It will be understood that the application of thrombin and fibrinogen or any other two-components for the bonding agent could be reversed in order. As well, the components of the bonding agents can be applied by any suitable method, including spray or brush application methods, and intermediate constructs can be hydrated at appropriate points in the manufacture. The entire construct may then be freeze-dried or otherwise processed if desired.

Submucosa for use in the invention can be derived from any suitable organ or other biological structure, including for example submucosa tissues derived from the alimentary, respiratory, intestinal, urinary or genital tracts of warm-blooded vertebrates. Submucosa useful in the present invention can be obtained by harvesting such tissue sources and delaminating the submucosa from smooth muscle layers, mucosal layers, and/or other layers occurring in the tissue source. For additional information as to submucosa useful in the present invention, and its isolation and treatment, reference can be made to U.S. Pat. Nos. 4,902,508, 5,554,389, 5,993,844, 6,206,931, and 6,099,567.

As prepared and used, the juvenile submucosa tissue and any other tissue used, may optionally retain growth factors or other bioactive components native to the source tissue. For example, the submucosa or other tissue may include one or more growth factors such as basic fibroblast growth factor (FGF-2), transforming growth factor beta (TGF-beta), epidermal growth factor (EGF), and/or platelet derived growth factor (PDGF). As well, submucosa tissue used in the invention may include other biological materials such as heparin, heparin sulfate, hyaluronic acid, fibronectin and the like. Thus, generally speaking, the submucosa or other tissue may include a bioactive component that induces, directly or indirectly, a cellular response such as a change in cell morphology, proliferation, growth, protein or gene expression. Further, in addition or as an alternative to the inclusion of such native bioactive components, non-native bioactive components such as those synthetically produced by recombinant technology or other methods, may be incorporated into the submucosa tissue.

Submucosa tissue used in the invention is preferably highly purified, for example, as described in U.S. Pat. No. 6,206,931 to Cook et al. Thus, preferred material will exhibit an endotoxin level of less than about 12 endotoxin units (EU) per gram, more preferably less than about 5 EU per gram, and most preferably less than about 1 EU per gram. As additional preferences, the submucosa material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa tissue taught in U.S. Pat. No. 6,206,931 may be characteristic of the submucosa tissue used in the present invention.

A typical layer thickness for the as-isolated-juvenile submucosa layer used in the invention ranges from about 50 to about 200 microns when fully hydrated. This layer thickness may vary with the type and age of the animal used as the tissue source. As well, this layer thickness may vary with the source of the tissue obtained from the animal source. In particular, when juvenile mammalian submucosa tissue is used in the invention, the as-isolated submucosa layer will typically have a thickness in the range of about 80 to about 150 microns when fully hydrated, and the tissue will be generally more compliant than adult tissue from the same species. Other characteristics of juvenile small intestinal submucosa include, for example, a native inner diameter of about 1 mm to about 12 mm, more typically about 3 mm to about 8 mm. The native juvenile submucosa tissue may also exhibit a higher level of solubility in urea or other similar collagen-degrading agents, evidencing a difference in composition likely relating at least in part to the maturity of the collagen. Porcine, ovine, or bovine submucosa tissues having these characteristics are preferred for use in the present invention, particularly porcine small intestinal submucosa.

Submucosa tissue used in the invention may be free of additional, non-native crosslinking, or may contain additional crosslinking. Such additional crosslinking may be achieved by photo-crosslinking techniques, by chemical crosslinkers, or by protein crosslinking induced by dehydration or other means. Chemical crosslinkers that may be used include for example aldehydes such as glutaraldehydes, diimides such as carbodiimides, e.g., 1-ethyl-3-(3-dimethylaminopropyl)carbodiimide hydrochloride, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

When additionally crosslinked, submucosa tissues of the invention can be additionally crosslinked internally within a single layer, and/or crosslinking may be used in whole or in part to bond multiple submucosa layers to one another. Thus, additional crosslinking may be added to individual submucosa layers prior to bonding to one another, during bonding to one another, and/or after bonding to one another.

Graft constructs in accordance with the invention can be used to graft mammalian patients, including humans. Preferred, tubular graft constructs of the invention find particular utility in repairing or replacing tubular structures within the body. For example, tubular graft constructs of the invention are used with preference in vascular applications nerve tube applications, ductal repair or replacement, urethral repair or replacement, or ureter repair or replacement. Vascular applications include, for example, use as arterial or venous grafts, and/or bypass grafts. Generally, tubular graft constructs of the invention will have internal diameters ranging from about 1 mm to about 30 mm, more typically in the range of about 1 mm to about 12 mm, and most typically in the range of about 3 mm to about 8 mm.

Graft constructs of the invention may include coatings or other incorporated materials to assist in reducing the frequency or incidence of thrombosis when used in the vasculature. For example, grafts in the invention may be coated with heparin. In this regard, the heparin may be bound to the graft construct by any suitable method including physical, ionic, or covalent bonding. In one preferred embodiment, heparin is bound to the collagen construct using a suitable crosslinking agent such as a polyepoxide as described hereinabove. In multi-layer constructs, heparin or other agents can be applied to the layers individually before incorporation of the layer into the construct, after the layers are incorporated into the construct (e.g. coating a luminal surface of an inner tubular layer), or both.

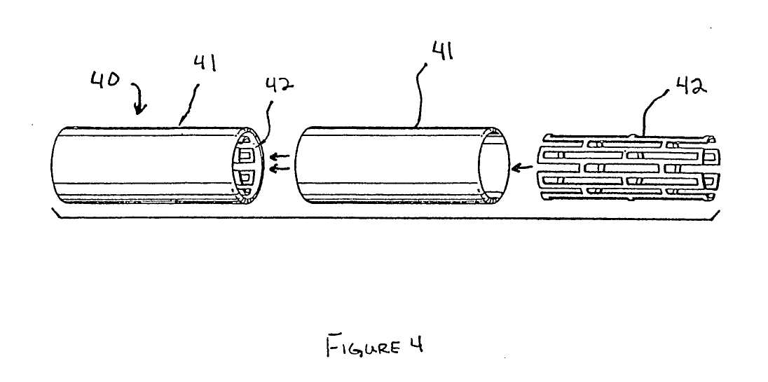

Prosthesis devices of the invention may optionally include medical structures other than tissue and/or polymer layers. For example, tissue graft materials including juvenile or other small-diameter submucosa may be attached or otherwise mounted in combination with stents, rings, valves, or other similar medical structures. In such devices, the tissue graft material may for example be used as a coating to facilitate tissue incorporation of the medical structure, and/or may be used to create one or more functional tissue segments, such as tissue valve structures, associated with the medical structure. With reference to FIG. 4, in certain embodiments of the invention, the tissue graft material including juvenile or other small diameter submucosa is used as a coating 41 for a stent 42 comprised of wire or another biocompatible material, to form a coated or sleeved stent device 40. In such devices, an intact cylindrical segment of juvenile or other small diameter submucosa provides an effective cylindrical coating or sleeve 41 for the stent 42. In this regard, cylindrical coating 41 may comprise a single layer or multilaminate construct in configurations described hereinabove, or still other configurations, and may optionally be attached to the stent 42 at one or more locations along its length, for example by sutures, adhesives, bonding, or other attachment means. The stent 41 may be a self-expanding stent, or an expandable stent (e.g. by balloon), useful for example in vascular, gastrointestinal, or other body passageways. In one mode of construction, the coating 41 may be applied to the stent 42 while the stent 42 is in its expanded state, and the stent 42 thereafter converted to a retracted state, preferably without causing significant damage to the coating 41. The coating 41 may optionally be in hydrated condition during retraction to facilitate this operation. The coated stent 40 can thereafter be conventionally processed and packaged for medical use.

The invention also encompasses medical products including a prosthesis device of the invention sealed within sterile medical packaging. The final, packaged product is provided in a sterile condition. This may be achieved, for example, by gamma, e-beam or other irradiation techniques, ethylene oxide gas, or any other suitable sterilization technique, and the materials and other properties of the medical packaging will be selected accordingly.

For the purposes of promoting an additional understanding of the invention and its features and advantages, the following specific examples are provided. It will be understood that these examples are illustrative, and not limiting, of the invention.

### EXAMPLE 1

### Isolation of Juvenile Intestinal Submucosa

Frozen intact juvenile porcine small intestine was immersed in tap (<38° C.) water until it was thawed. At room temperature, the intestine was then cleaned out by running tap water through the entire length to remove any remaining chyme. Then, the intestine was cut into one-foot lengths and hand-scraped with a Teflon plate. Scraped tubular submucosa pieces were placed into high purity water to keep hydrated until the material could be disinfected.

### EXAMPLE 2

### Disinfection of Juvenile Intestinal Submucosa

Isolated juvenile porcine small intestine submucosa was submerged into one liter of 0.2% (v/v) peracetic acid/0.05% (v/v) ethanol solution and was shaken for two hours at room temperature. After two hours, the peracetic acid solution was drained, and high purity water was added. The submucosa was then shaken at room temperature for five minutes and drained. Subsequently, the juvenile submucosa was rinsed three more times at room temperature with high purity water. Finally, the intact tubular juvenile submucosa was stored in high purity water at 4° C.

### EXAMPLE 3

### Preparation of Multilaminate Tubular Graft Construct

The luminal juvenile submucosal layer was pretreated with an antithrombogenic heparin coating. Then, the treated mucosal surface was positioned on a stainless steel mandrel so that the mandrel faced the lumen. The outer serosal surface was sprayed with the thrombin component of fibrin glue, while another piece of juvenile submucosa was inverted on another stainless steel mandrel, so that the serosal surface was contacting the mandrel. This piece was sprayed with the fibrinogen component. The ends of the two pieces were secured, and the inverted juvenile submucosa was reinverted onto the first layer. The fibrin glue was allowed to cure for two minutes at room temperature and submerged in high purity water. A sheet of hydrated adult small intestinal submucosa was laid out and completely sprayed with thrombin, and the outer surface of the two-layer graft was sprayed with fibrinogen. Then, the tubular submucosa was carefully laid on the adult submucosa and wrapped once with the adult submucosa. The remainder of the sheet was covered with Parafilm, and the outer surface of the third layer was sprayed with fibrinogen. One complete turn of the mandrel was made, and the excess adult submucosa was trimmed. The fibrin glue was allowed to cure for two minutes at room temperature, and the entire four-layer graft was submerged into high purity water for two minutes. A final layer of juvenile submucosa was inverted onto another stainless steel mandrel and sprayed with thrombin. The four-layer graft was then sprayed with fibrinogen. The final juvenile submucosa layer was reinverted onto the outer surface of the four-layer graft and allowed to cure for two minutes at room temperature. The graft was submerged back into high purity water for thirty minutes, then frozen for three minutes in a −80° C. freezer. Finally, the graft was lyophilized overnight, trimmed, packaged, and sterilized via ethylene oxide sterilization.

### EXAMPLE 4

### Utilization of Multilaminate Graft Construct

A five-layer multi-laminate small diameter vascular graft containing multiple layers of tubular juvenile small intestinal submucosa was implanted into the left anterior descending coronary artery of a 70-pound dog. The graft was anastomosed in with a running 7-0 prolene suture, with each suture spaced about 1-2 mm apart. Upon removal of the clamps, normal blood flow was re-established, and the graft was patent with no apparent leaking from the anastomosis or dilatation.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all publications cited herein are hereby incorporated by reference in their entirety as if each had been individually incorporated by reference and fully set forth.

What is claimed is:

1. A tubular graft construct, comprising:
a tubular element having walls and a lumen; and
said walls including juvenile submucosa tissue from a warm-blooded vertebrate.

2. A tubular graft construct of claim 1, wherein said juvenile submucosa tissue retains a natural, intact tubular form.

3. A tubular graft construct of claim 2, wherein said juvenile submucosa tissue is juvenile small intestinal submucosa tissue, and wherein a surface of said lumen is defined by said juvenile small intestinal submucosa tissue.

4. A tubular graft construct of claim 1, wherein said walls include a layer provided by said juvenile submucosa tissue, and at least one additional layer.

5. A tubular graft construct of claim 4, wherein a surface of said lumen is defined by said submucosa tissue.

6. A tubular graft construct of claim 5, wherein said at least one additional layer includes a collagen layer.

7. A tubular graft construct of claim 6, wherein said collagen layer is a naturally derived collagen layer.

8. A tubular graft construct of claim 7, wherein said naturally derived collagen layer is an extracellular matrix layer.

9. A tubular graft construct of claim 5, wherein said at least one additional layer includes a synthetic layer.

10. A tubular graft construct of claim 9, wherein said synthetic layer is comprised of a synthetic polymer.

11. A tubular graft construct of claim 8, wherein said extracellular matrix layer comprises submucosa, dura mater, pericardium, serosa, peritoneum, or basement membrane.

12. A tubular graft construct of claim 11, wherein said extracellular matrix layer comprises submucosa.

13. A tubular graft construct of claim 12, wherein said submucosa is mammalian submucosa.

14. A tubular graft construct of claim 13, wherein said mammalian submucosa is porcine, bovine, or ovine submucosa.

15. A tubular graft construct of claim 14, wherein said submucosa is porcine submucosa.

16. A tubular graft construct of claim 15, wherein said porcine submucosa is adult porcine submucosa.

17. A tubular graft construct of claim 15, wherein said porcine submucosa is a second layer of juvenile porcine submucosa.

18. A tubular graft construct according to claim 17, wherein said second layer of juvenile porcine submucosa constitutes an outermost layer of the construct.

19. A tubular graft construct of claim 18, comprising:
an innermost layer defining a surface of the lumen, the innermost layer provided by juvenile small intestinal submucosa tissue retaining a natural, intact tubular form;
at least one intermediate layer; and
an outermost layer provided by juvenile small intestinal submucosa tissue retaining a natural, intact tubular form.

20. A tubular graft construct of claim 19, wherein said at least one intermediate layer includes a collagenous layer.

21. A tubular graft construct of claim 20, wherein said collagenous layer is an extracellular matrix layer.

22. A tubular graft construct of claim 21, wherein said extracellular matrix layer is submucosa.

23. A tubular graft construct of claim 22, wherein said submucosa is small intestinal submucosa.

24. A tubular graft construct of claim 23, wherein said small intestinal submucosa is adult small intestinal submucosa.

25. A tubular graft construct of claim 24, wherein said adult small intestinal submucosa is porcine small intestinal submucosa.

26. A tubular graft construct of claim 4, wherein said juvenile submucosa layer and at least one additional layer are bonded to one another.

27. A tubular graft construct of claim 19, wherein said innermost layer, at least one intermediate layer, and outermost layer are bonded to one another.

28. A tubular graft construct of claim 1, wherein said lumen has an anti-thrombogenic coating.

29. A tissue graft composition, comprising juvenile submucosa tissue.

30. A tissue graft composition of claim 29, wherein said submucosa tissue is small intestinal submucosa tissue.

31. A tissue graft composition of claim 30, wherein said small intestinal submucosa tissue retains an intact, tubular form.

32. A tissue graft composition of claim 31, wherein said tubular form has a diameter not exceeding about 8 mm.

33. A tissue graft composition of claim 29, wherein said submucosa tissue is porcine, bovine, or ovine.

34. A method for tissue grafting in a mammal, comprising grafting said mammal with a tissue graft material comprising juvenile submucosa tissue.

35. A method of claim 34, wherein said juvenile submucosa tissue retains an intact, tubular form.

36. A method of claim 35, wherein said tubular form has a diameter not exceeding about 12 mm.

37. A method of claim 34, wherein said juvenile submucosa tissue is porcine, bovine, or ovine.

38. A tubular graft construct, comprising:
a tubular element having walls and a lumen;
said walls including at least a first layer formed with intact tubular submucosa having a native internal diameter no greater than about 12 mm.

39. A tubular graft construct of claim 38, wherein said walls include at least a second layer.

40. A tubular graft construct of claim 39, wherein said second layer includes submucosa tissue.
